(19) United States
(12) Patent Application Publication
(10) Pub. No.: US 2008/0153881 A1
(43) Pub. Date: Jun. 26, 2008

(54) ALPHA-2B RECEPTOR AGONIST AND ACID REDUCER COMPOSITIONS FOR TREATING GASTROINTESTINAL MOTILITY DISORDERS

(75) Inventors: Gregory F. Brooks, Irvine, CA (US); Daniel W. Gil, Corona Del Mar, CA (US)

Correspondence Address: ALLERGAN, INC., 2525 DUPONT DRIVE, T2-7H, IRVINE, CA, 92612-1599, US

(73) Assignee: Allergan, Inc.

(21) Appl. No.: 11/954,530

(22) Filed: Dec. 12, 2007

Related U.S. Application Data
(60) Provisional application No. 60/871,706, filed on Dec. 22, 2006.

(51) Int. Cl. A61K 31/4439; A61K 31/341; A61K 31/4164; A61P 1/00; A61K 31/426

(52) U.S. Cl. 514/338; 514/400; 514/370; 514/471; 514/365

(57) ABSTRACT

Disclosed herein is a pharmaceutical composition comprising an acid reducer and an alpha-2B receptor agonist. The composition is effective for treating gastrointestinal motility disorders, and methods of treating such disorders using the composition and compounds comprising it are also disclosed.

### CROSS REFERENCE TO RELATED APPLICATIONS

This application is based on, and claims the benefit of, U.S. Provisional Application No. 60/871,706, filed Dec. 22, 2006, and which is incorporated herein by reference.

Disclosed herein is a pharmaceutical composition comprising an acid reducer and an alpha-2B receptor agonist. The composition is effective for treating gastrointestinal motility disorders, and methods of treating such disorders using the composition and compounds comprising it are also disclosed. Administering an alpha-2B receptor agonist together with an acid reducer increases the efficacy of these compounds in treating the gastrointestinal motility disorder.

### DETAILED DESCRIPTION OF THE INVENTION

### Disorders of Gastrointestinal Motility

“Gastrointestinal motility” refers to the movement of food through the gastrointestinal tract. A “disorder of gastrointestinal motility” is any abnormality in that process that causes discomfort to a patient. It includes, for example, achalasia, Barrett's syndrome, biliary dyskinesia, Crohn's disease, chronic intestinal pseudo-obstruction, colonic inertia, constipation, cyclic vomiting syndrome, diarrhea, diffuse esophageal spasm, dumping syndrome, dyspepsia, dysphagia, encopresis, fecal incontinence, functional abdominal pain (e.g., chronic proctalgia, epigastric pain syndrome, functional abdominal pain syndrome, proctalgia fugax), functional biliary disorders (e.g., functional biliary SO disorder, functional gallbladder disorder, functional pancreatic SO disorder, functional sphincter of Oddi disorder), functional bowel outlet obstruction, functional dyspepsia disorders (e.g., epigastric pain syndrome, functional dyspepsia, postprandial distress syndrome), functional esophogeal disorders (e.g., functional chest pain of presumed esophogeal origin, functional dysphagia, functional heartburn, globus), functional fecal retention, gastroesophageal reflux disease (GERD), gastroparesis, gastritis, gastropathy, Hirschprung's disease, hypercontractile motility, hypermotility, hypertensive lower esophageal sphincter, hypomotility, intestinal obstruction, irritable bowel syndrome, ischemia, megacolon, non-erosive reflux disease, pancreatitis, pelvic floor dysfunction, short bowel syndrome, small bowel bacterial overgrowth, small bowel intestinal motility disorder, superior mesenteric artery syndrome, ulcerative colitis, and volvulus.

It also includes any symptom produced by disorders of gastrointestinal motility that results in discomfort to a patient, regardless of how one would categorize the disorder that creates the discomfort. Hence, “disorder of gastrointestinal motility” also includes, for example, altered bowel habit (including, for example, change in stool frequency; change in stool form, such as passing hard or loose stools; or change in the manner of passing stool, such as straining, urgency, or feeling or incomplete evacuation), belching, bloating (including a feeling of abdominal distension), blood or mucus in the stool, diarrhea, dyspepsia, dysphagia, flatulence, globus, hoarseness of voice, loss of appetite, nausea, pain in any area or the chest, colon, stomach, or elsewhere in the abdomen, pyrosis (heartburn), regurgitation, sore throat, trapped gas, and uncomfortable fullness after meals.

### Acid Reducers

Acid reducers are agents that neutralize gastric acid or decrease the stomach's production of it. Acid reducers useful in the method of the invention include, for example, antacids, hydrogen-potassium ATPase inhibitors (also known as proton pump inhibitors), and histamine H₂ receptor antagonists.

### Antacids

Antacids are compounds which react with hydrochloric acid, the principal component of gastric acid, to form salt and water. Antacids are well known in the art and are described, for example, in Remington, The Science and Practice of Pharmacy, Vol. II, Nineteenth Edition, 886-890 (1995).

Antacids include, for example, aluminum salts, bismuth salts, calcium carbonate, magnesium salts, potassium bicarbonate, potassium citrate, sodium bicarbonate, sodium potassium tartrate, tricalcium phosphate, and mixtures of any of the foregoing.

Aluminum salts include, for example, alexitol sodium (aluminum sodium carbonate hexitol complex), almagate (carbonic acid, aluminum magnesium complex), aluminum hydroxide, aluminum magnesium silicate, aluminum phosphate, basic aluminum carbonate gel (aluminum hydroxide-aluminum carbonate gel), sucralfate (basic aluminum sucrose sulfate complex), dihydroxyaluminum aminoacetate, dihydroxyaluminum sodium carbonate, and magaldrate (aluminum magnesium hydroxide monohydrate).

Bismuth salts include, for example, bismuth aluminate, bismuth phosphate, bismuth carbonate, bismuth subcarbonate, bismuth subgallate, and bismuth subnitrate.

Magnesium salts include, for example, magnesium carbonate, magnesium hydroxide, magnesium oxide, magnesium peroxide, magnesium phosphate, tribasic magnesium silicates (magnesium trisilicate), and magnesium aluminosilicates.

Other salts of bicarbonate, citrate, phospate, and tartrate include, for example, sodium bicarbonate, potassium bicarbonate, potassium citrate, sodium potassium tartrate, and tricalcium phosphate.

### Proton Pump Inhibitors

Proton pump inhibitors inhibit parietal cells from secreting H⁺ into the gastric lumen by inhibiting the H⁺/K⁺ ATPase enzyme system at the secretary surface of the cell. Examples of proton pump inhibitors include esomeprazole, lansoprazole omeprazole, pantoprazole, rabeprazole, and other benzoimidazoles.

Esomeprazole is a proton pump inhibitor having the following structure:

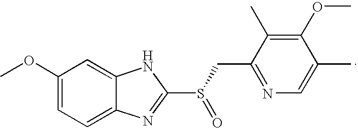

### The magnesium salt of esomeprazole is sold in the United States under the brand name Nexium®.

Lansoprazole is a proton pump inhibitor having the following structure:

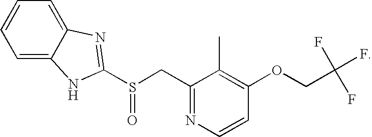

### Lansoprazole is sold in the United States under the brand name Prevacid®.

Omeprazole is a proton pump inhibitor having the following structure:

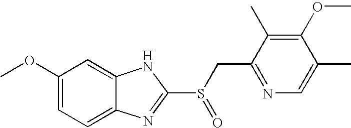

### The magnesium salt of omeprazole is sold in the United States under the brand name Prilosec®. It is a racemic mixture; esomeprazole is an enantiomer of omeprazole.

Pantoprazole is a proton pump inhibitor having the following structure:

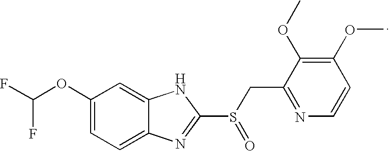

### The sodium salt of pantoprazole is sold in the United States under the brand name Protonix®.

Rabeprazole is a proton pump inhibitor having the following structure:

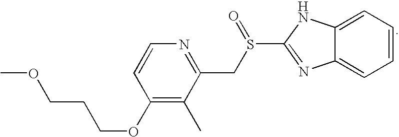

### The sodium salt of rabeprazole is sold in the United States under the brand name Aciphex®.

### Histamine H₂ Receptor Antagonists

Histamine H₂ receptor antagonists prevent histamine from binding to histamine H₂ receptors on parietal and other cells, decreasing acid production by parietal cells. Examples of histamine H₂ receptor antagonists include cimetidine, famotidine, nizatidine, and ranitidine.

Cimetidine is a histamine H₂ receptor antagonists having the following structure:

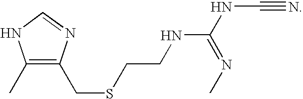

### Cimetidine is sold in the United States under the brand name Tagamet®.

Famotidine is a histamine H₂ receptor antagonists having the following structure:

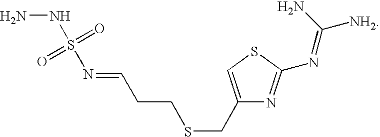

### Famotidine is sold in the United States under the brand name Pepcid®.

Nizatidine is a histamine H₂ receptor antagonists having the following structure:

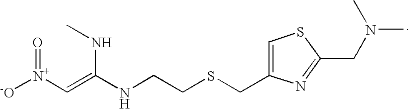

### Nizatidine is sold in the United States under the brand name Axid®.

Ranitidine is a histamine H₂ receptor antagonists having the following structure:

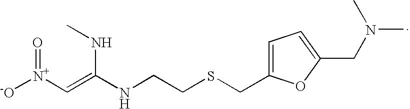

### The hydrochloride salt of ranitidine is sold in the United States under the brand name Zantac®.

### Pharmaceutically Acceptable Salts

One can use in the compositions and methods of the invention any acid reducer as its pharmaceutically acceptable salt.

A “pharmaceutically acceptable salt” is any salt that retains the activity of the parent compound and does not impart any additional deleterious or untoward effects on the subject to which it is administered and in the context in which it is administered compared to the parent compound. A pharmaceutically acceptable salt also refers to any salt which may form in vivo as a result of administration of an acid, another salt, or a prodrug which is converted into an acid or salt.

Pharmaceutically acceptable salts of acidic functional groups may be derived from organic or inorganic bases. The salt may comprise a mono or polyvalent ion. Of particular interest are the inorganic ions lithium, sodium, potassium, calcium, and magnesium. Organic salts may be made with amines, particularly ammonium salts such as mono-, di- and trialkyl amines or ethanol amines. Salts may also be formed with caffeine, tromethamine and similar molecules. Hydrochloric acid or some other pharmaceutically acceptable acid may form a salt with a compound that includes a basic group, such as an amine or a pyridine ring.

### Prodrugs

One can use in the compositions and methods of the invention a prodrug of any acid reducer.

A “prodrug” is a compound which is converted to a therapeutically active compound after administration, and the term should be interpreted as broadly herein as is generally understood in the art. While not intending to limit the scope of the invention, conversion may occur by hydrolysis of an ester group or some other biologically labile group. Generally, but not necessarily, a prodrug is inactive or less active than the therapeutically active compound to which it is converted. Ester prodrugs of the compounds disclosed herein are specifically contemplated. An ester may be derived from a carboxylic acid of C1 (i.e., the terminal carboxylic acid of a natural prostaglandin), or an ester may be derived from a carboxylic acid functional group on another part of the molecule, such as on a phenyl ring. While not intending to be limiting, an ester may be an alkyl ester, an aryl ester, or a heteroaryl ester. The term alkyl has the meaning generally understood by those skilled in the art and refers to linear, branched, or cyclic alkyl moieties. C₁₋₆ alkyl esters are particularly useful, where alkyl part of the ester has from 1 to 6 carbon atoms and includes, but is not limited to, methyl, ethyl, propyl, isopropyl, n-butyl, sec-butyl, iso-butyl, t-butyl, pentyl isomers, hexyl isomers, cyclopropyl, cyclobutyl, cyclopentyl, cyclohexyl, and combinations thereof having from 1-6 carbon atoms, etc.

The acid reducers and alpha-2B receptor agonists of the invention may be either synthetically produced, or may be produced within the body after administration of a prodrug. Hence, “acid reducer” and “alpha-2B receptor agonist” encompass compounds produced by a manufacturing process and those compounds formed in vivo only when another drug administered.

### Isomers and Racemates

One can use in the compositions and methods of the invention an enantiomer, stereoisomer, or other isomer of any acid reducer.

### Alpha-2B Adrenergic Receptor Agonists

Alpha-2B adrenergic receptor agonists are those compounds that activate to the alpha-2B adrenergic receptor subtype. A compound is an “alpha-2B receptor agonist” if it has greater than 25% efficacy relative to brimonidine at the alpha-2B adrenergic receptor. A compound need not be selective for the alpha-2B adrenergic receptor to be an alpha-2B receptor agonist: the term encompasses agonists that activate alpha-2 adrenergic receptor subtypes other than the alpha-2B receptor subtype and that activate alpha-1 adrenergic receptor subtypes, as well; all such agonists are “alpha-2B receptor agonists” provided that they have greater than 25% efficacy relative to brimonidine at the alpha-2B receptor subtype.

One can use in the compositions and methods of the invention alpha-2B receptor agonists that are also alpha-2C receptor agonists. A compound is an “alpha-2C receptor agonist” if it has greater than 25% efficacy relative to brimonidine at the alpha-2C receptor. Such an agonist can also be an alpha-2B receptor agonist—an “alpha 2B/2C receptor agonist”—if it also has greater than 25% efficacy relative to brimonidine at the alpha-2B receptor subtype. Note that an agonist can activate the alpha-2C receptor subtype and yet not have 25% efficacy relative to brimonidine at that subtype; such agonists can still be “alpha-2B receptor agonists,” yet are not “alpha-2B/2C receptor agonists” as those terms are defined here.

One can also use in the compositions and methods of the invention alpha-2B receptor agonists lacking significant activity at the alpha-2A receptor subtype. An agonist lacks significant alpha-2A receptor activity if the agonist has less than 40% of the efficacy of brimonidine at the alpha-2A receptor subtype. The invention therefore includes, for example, alpha-2B receptor agonists lacking significant alpha-2A activity; alpha 2B/2C receptor agonists lacking significant alpha-2A activity; and alpha-2B receptor agonists, lacking significant alpha-2A activity, that activate one or more alpha-1 adrenergic receptor subtypes.

Efficacy, also known as intrinsic activity, is a measure of maximal receptor activation achieved by a compound and can be determined using any accepted assay of alpha-adrenergic receptor activation, such as a cAMP or Receptor Selection and Amplification Technology (RSAT). Efficacy is represented as a ratio or percentage of the maximal effect of the drug to the maximal effect of a standard agonist for each receptor subtype. Brimonidine, itself an alpha-2B receptor agonist (it is has 100% the efficacy of brimonidine at the alpha-2B adrenergic receptor), is used as the standard agonist for the alpha-2B adrenergic receptors.

Agonist activity can be characterized using any of a variety of routine assays, including, for example, Receptor Selection and Amplification Technology (RSAT) assays (Messier et al., Pharmacol. Toxicol. 76:308-11 (1995); cyclic AMP assays (Shimizu et al., J. Neurochem. 16:1609-1619 (1969)); and cytosensor microphysiometry assays (Neve et al., J. Biol. Chem. 267:25748-25753 (1992)). Such assays generally are performed using cells that naturally express only a single alpha-adrenergic receptor subtype, or using transfected cells expressing a single recombinant alpha-adrenergic receptor subtype. The adrenergic receptor can be a human receptor or homolog of a human receptor having a similar pharmacology.

The RSAT assay measures receptor-mediated loss of contact inhibition resulting in selective proliferation of receptor-containing cells in a mixed population of confluent cells. The increase in cell number is assessed with an appropriate detectable marker gene such as beta-galactosidase, if desired, in a high throughput or ultra high throughput assay format. Receptors that activate the G protein, Gq, elicit the proliferative response. Alpha-adrenergic receptors, which normally couple to Gi, activate the RSAT response when coexpressed with a hybrid Gq protein containing a Gi receptor recognition domain, designated Gq/i5. Conklin et al., Nature 363:274-6 (1993)).

As an example, an RSAT assay can be performed essentially as follows. NIH-3T3 cells are plated at a density of 2×10⁶ cells in 15 cm dishes and maintained in Dulbecco's modified Eagle's medium supplemented with 10% calf serum. One day later, cells are cotransfected by calcium phosphate precipitation with mammalian expression plasmids encoding p-SV-β-galactosidase (5-10 μg), receptor (1-2 μg) and G protein (1-2 μg). Carrier DNA, for example 40 μg salmon sperm DNA, also can be included to increase transfection efficiency. Fresh media is added on the following day; one to two days later, cells are harvested and frozen in 50 assay aliquots. Transfected cells are thawed, and 100 μl of cells added to 100 μl aliquots of compound to be tested, with various concentrations assayed in triplicate, for example, in 96-well plates. Incubation continues for 72 to 96 hours at 37° C. After washing with phosphate-buffered saline, β-galactosidase activity is determined by adding 200 μl of chromogenic substrate (3.5 mM O-nitrophenyl-β-D-galactopyranoside/0.5% NP-40 in phosphate buffered saline), incubating overnight at 30° C., and measuring optical density at 420 nm. The absorbancy is a measure of enzyme activity, which depends on cell number and reflects receptor-mediated cell proliferation. The EC₅₀ and maximal effect (i.e., efficacy) of each drug at each receptor is determined.

Exemplary alpha-2B receptor agonists include the compounds below in Table 1:

TABLE 1
Alpha-2B receptor agonists

| COM- | |
|---|---|
| POUND | STRUCTURE |
| 1 | 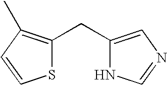 |
| 2 | 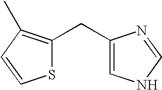 |
| 3 | 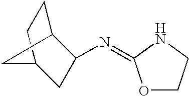 |
| 4 | 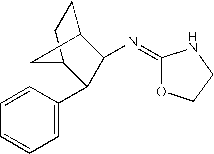 |
| 5 | 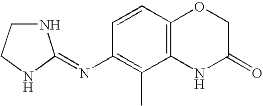 |
| 6 | 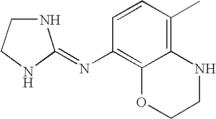 |
| 7 | 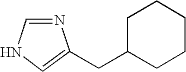 |
| 8 | 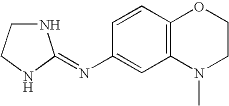 |
| 9 | 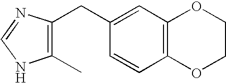 |
| 10 | 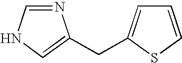 |
| 11 | 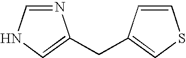 |
| 12 | 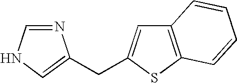 |
| 13 | 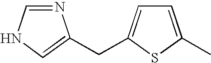 |
| 14 | 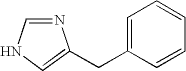 |
| 15 | 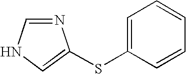 |
| 16 | 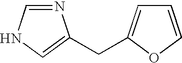 |
| 17 | 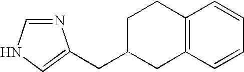 |
| 18 | 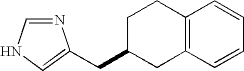 |
| 19 | 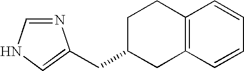 |
| 20 | 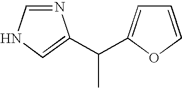 |
| 21 | 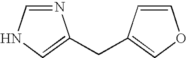 |
| 22 | 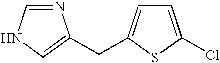 |
| 23 | 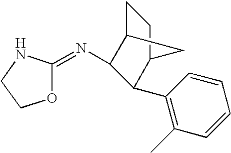 |
| 24 | 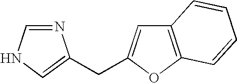 |
| 25 | 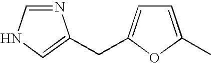 |
| 26 | 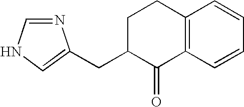 |
| 27 | 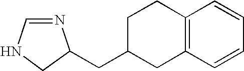 |
| 28 | 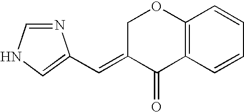 |
| 29 | 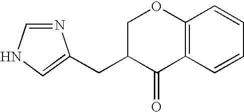 |
| 30 | 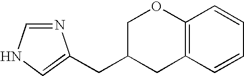 |
| 31 | 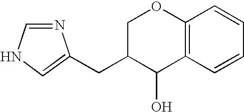 |
| 32 | 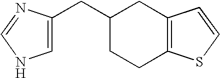 |
| 33 | 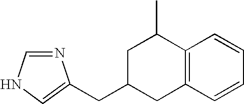 |
| 34 | 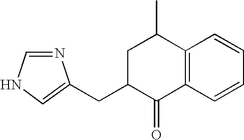 |
| 35 | 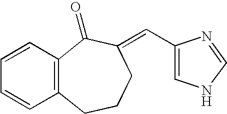 |
| 36 | 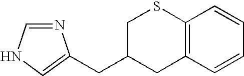 |
| 37 | 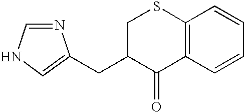 |
| 38 | 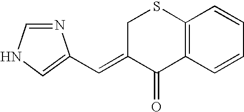 |
| 39 | 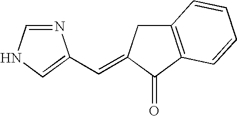 |
| 40 | 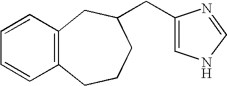 |
| 41 | 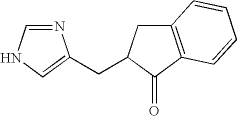 |
| 42 | 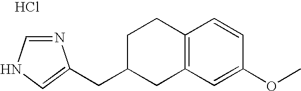 |
| 43 | 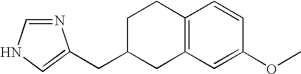 |
| 44 | 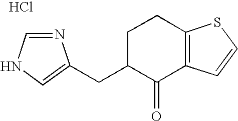 |
| 45 | 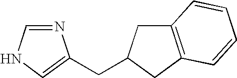 |
| 46 | 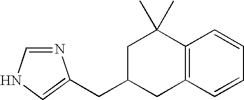 |
| 47 | 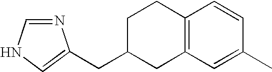 |
| 48 | 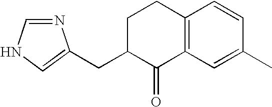 |
| 49 | 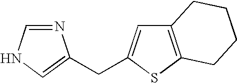 |
| 50 | 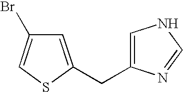 |
| 51 | 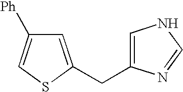 |
| 52 | 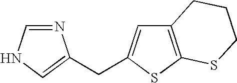 |
| 53 | 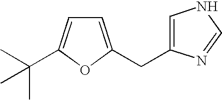 |
| 54 | 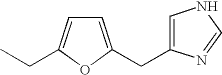 |
| 55 | 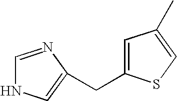 |
| 56 | 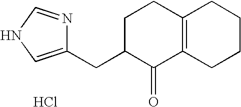 |
| 57 | 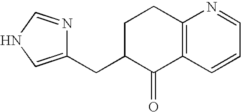 |
| 58 | 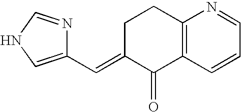 |
| 59 | 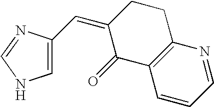 |
| 60 | 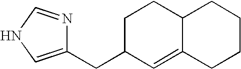 |
| 61 | 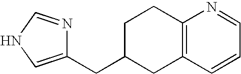 |
| 62 | 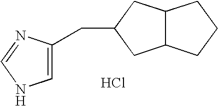 |
| 63 | 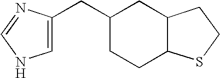 |
| 64 | 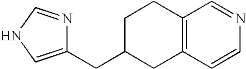 |
| 65 | 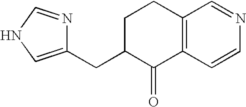 |
| 66 | 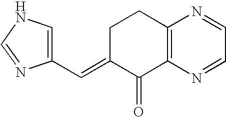 |
| 67 | 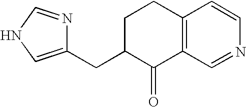 |
| 68 | 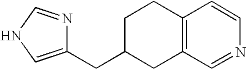 |
| 69 | 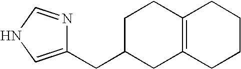 |
| 70 | 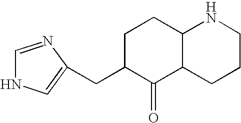 |
| 71 | 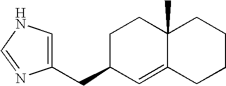 |
| 72 | 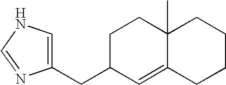 |
| 73 | 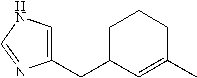 |
| 74 | 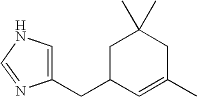 |
| 75 | 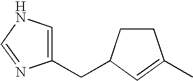 |
| 76 | 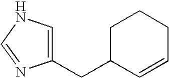 |
| 77 | 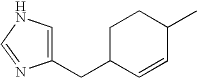 |
| 78 | 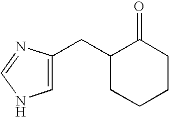 |
| 79 | 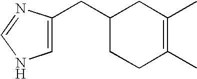 |
| 80 | 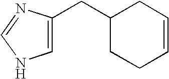 |
| 81 | 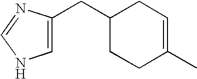 |
| 82 | 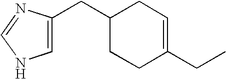 |
| 83 | 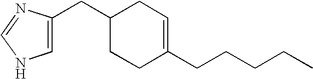 |
| 84 | 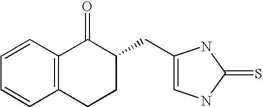 |
| 85 | 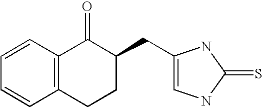 |
| 86 | 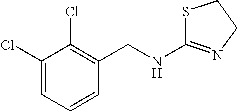 |
| 87 | 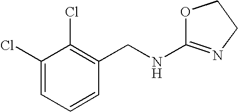 |
| 88 | 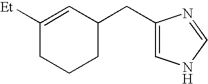 |
| 89 | 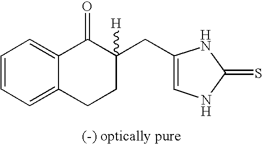 |
| 90 | 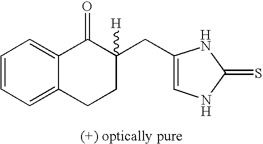 |
| 91 | 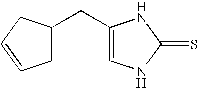 |
| 92 | 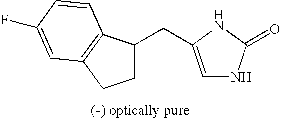 |
| 93 | 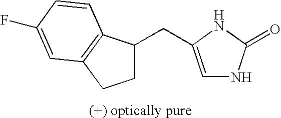 |
| 94 | 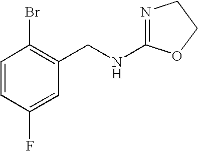 |
| 95 | 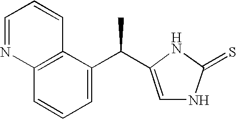 |
| 96 | 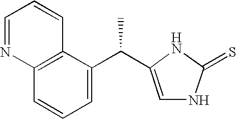 |
| 97 | 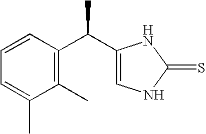 |
| 98 | 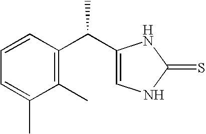 |
| 99 | 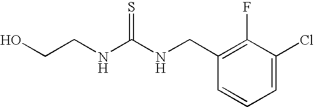 |
| 100 | 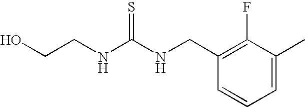 |
| 101 | 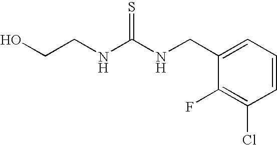 |
| 102 | 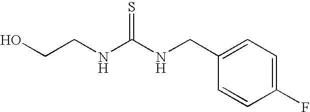 |
| 103 | 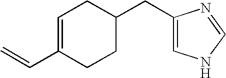 |
| 104 | 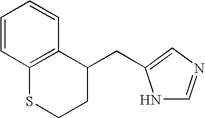 |
| 105 | 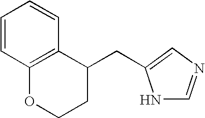 |
| 106 | 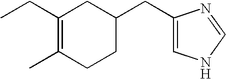 |
| 107 | 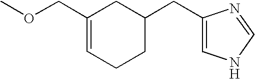 |
| 108 | 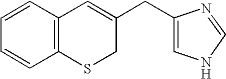 |
| 109 | 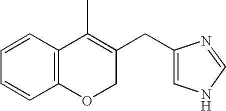 |
| 110 | 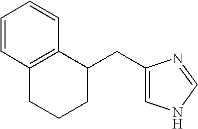 |
| 111 | 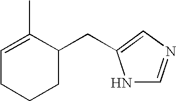 |
| 112 | 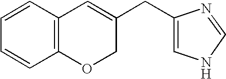 |
| 113 | 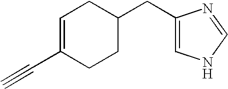 |
| 114 | 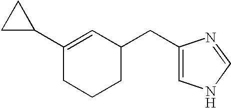 |
| 115 | 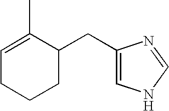 |
| 116 | 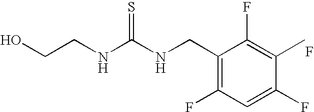 |
| 117 | 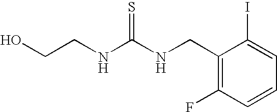 |
| 118 | 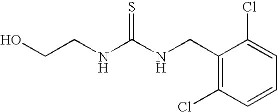 |
| 119 | 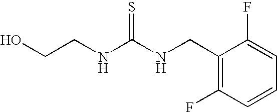 |
| 120 | 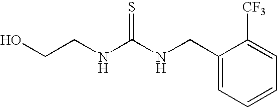 |
| 121 | 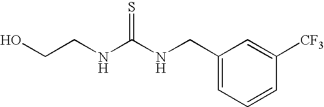 |
| 122 | 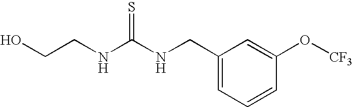 |
| 123 | 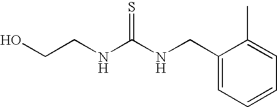 |
| 124 | 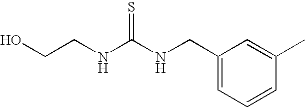 |
| 125 | 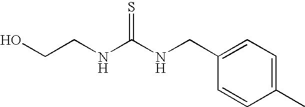 |
| 126 | 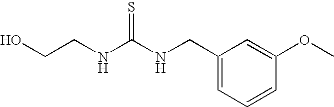 |
| 127 | 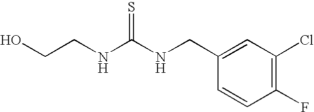 |
| 128 | 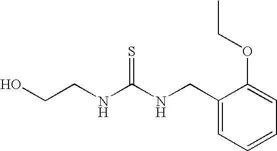 |
| 129 | 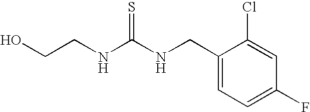 |
| 130 | 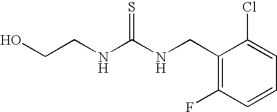 |
| 131 | 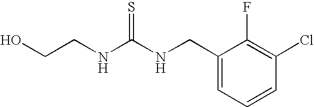 |
| 132 | 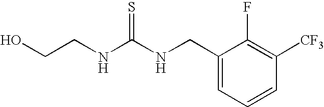 |
| 133 | 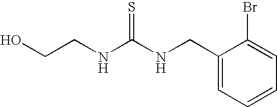 |
| 134 | 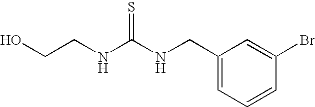 |
| 135 | 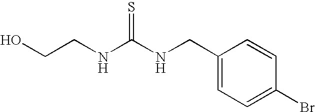 |
| 136 | 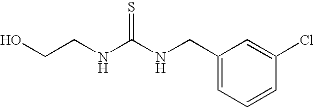 |
| 137 | 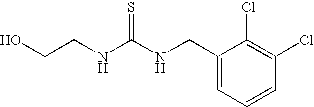 |
| 138 | 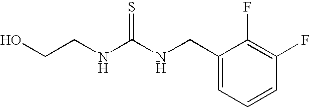 |
| 139 | 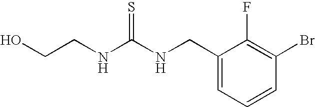 |
| 140 | 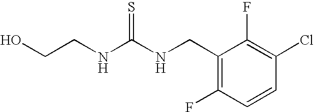 |
| 141 | 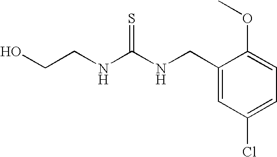 |
| 142 | 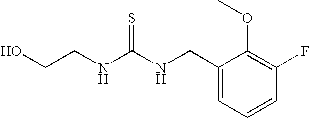 |
| 143 | 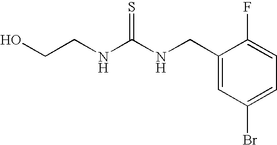 |
| 144 | 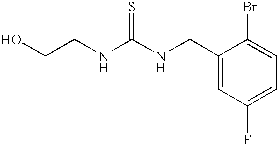 |
| 145 | 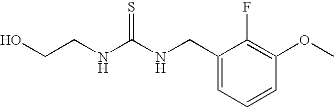 |
| 146 | 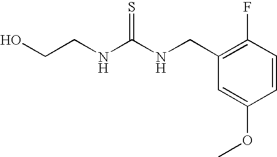 |
| 147 | 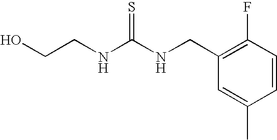 |
| 148 | 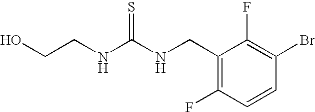 |
| 149 | 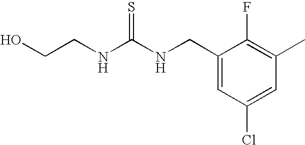 |
| 150 | 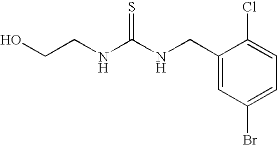 |
| 151 | 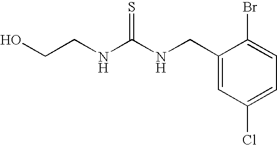 |
| 152 | 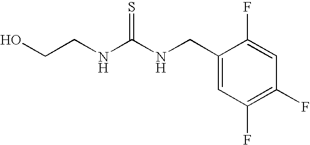 |
| 153 | 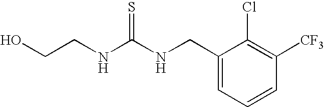 |
| 154 | 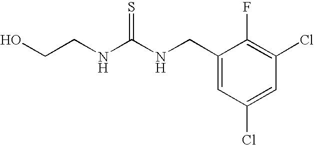 |
| 155 | 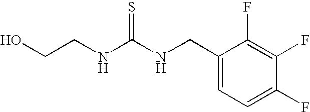 |
| 156 | 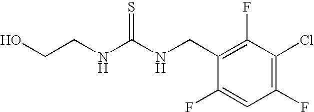 |
| 157 | 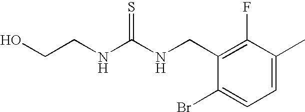 |
| 158 | 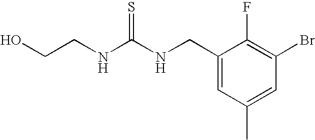 |
| 159 | 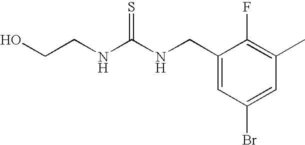 |
| 160 | 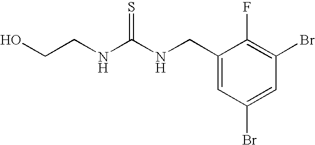 |
| 161 | 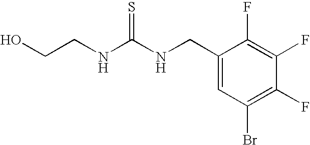 |
| 162 | 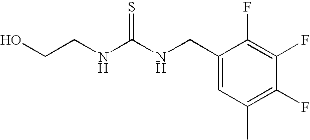 |
| 163 | 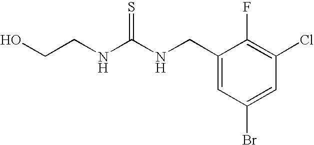 |
| 164 | 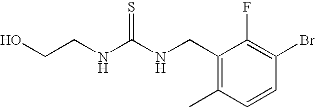 |
| 165 | 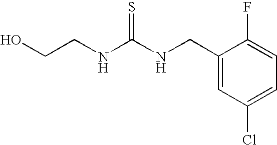 |
| 166 | 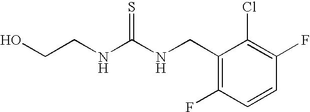 |
| 167 | 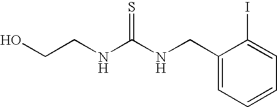 |
| 168 | 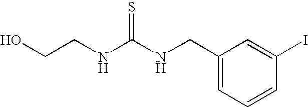 |
| 169 | 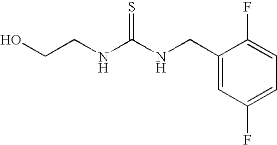 |
| 170 | 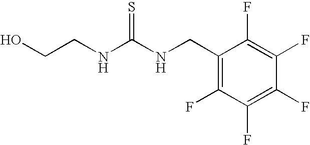 |
| 171 | 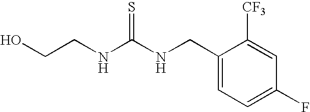 |
| 172 | 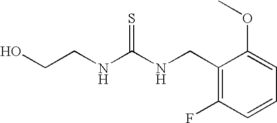 |
| 173 | 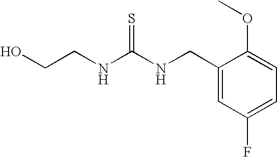 |
| 174 | 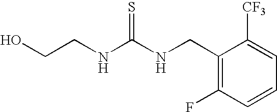 |
| 175 | 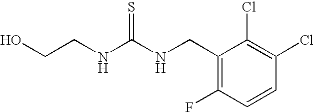 |
| 176 | 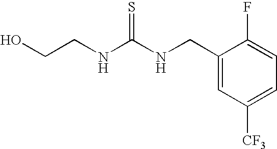 |
| 177 | 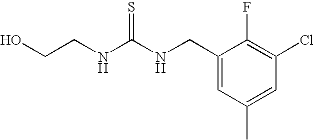 |
| 178 | 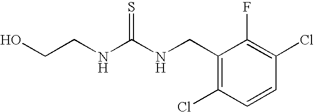 |
| 179 | 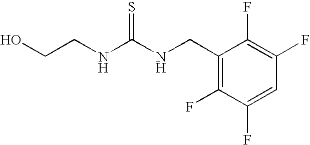 |
| 180 | 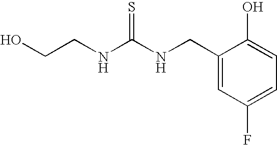 |
| 181 | 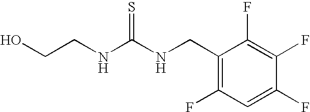 |
| 182 | 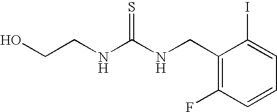 |
| 183 | 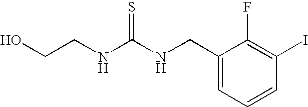 |
| 184 | 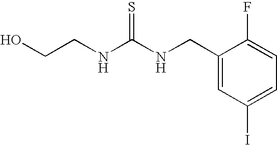 |
| 185 | 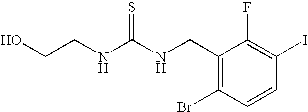 |
| 186 | 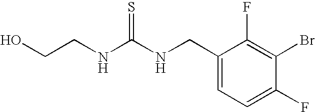 |
| 187 | 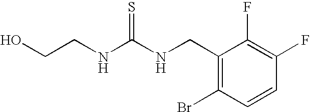 |
| 188 | 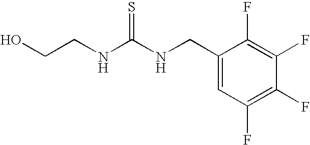 |
| 189 | 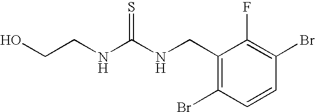 |
| 190 | 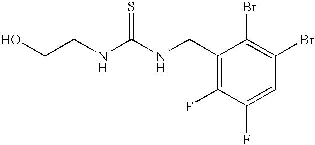 |
| 191 | 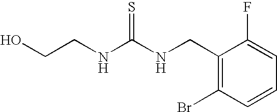 |
| 192 | 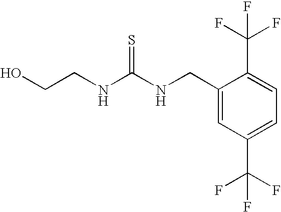 |
| 193 | 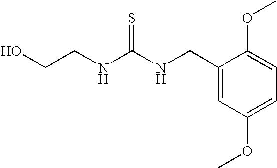 |
| 194 | 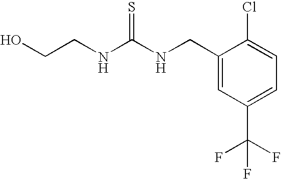 |
| 195 | 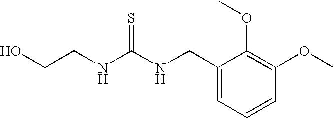 |

U.S. Pat. No. 6,329,369, U.S. Pat. No. 6,534,542, U.S. Pat. No. 6,545,182, U.S. Pat. No. 6,787,517, U.S. Pat. No. 6,841,684, and U.S. Pat. No. 7,091,232, and U.S. Patent Application Publication No. 2003/0092766, No. 2004/0132824, No. 2004/0220402, No. 2005/0075366, No. 2005/0267186, and U.S. patent application Ser. No. 11/172,229, Ser. No. 11/232,323, No. 11/232,341, No. 60/613,870, and No. 60/695,650, the disclosures of all which are incorporated herein by reference, provide additional information regarding alpha-2B receptor agonists.

One can use in the methods and compositions of the invention any pharmaceutically acceptable salt, prodrug, isomer, and racemate (as those terms are defined in the preceding sections) of any alpha-2B receptor agonist.

### Pharmaceutical Compositions

Pharmaceutical compositions of the invention comprise one or more acid reducer and one or more alpha-2B receptor agonist.

### Excipients and Dosage Forms

Those skilled in the art will readily understand that for administering pharmaceutical compositions of the invention acid reducers and alpha-2B receptor agonists can be admixed with pharmaceutically acceptable excipient which are well known in the art.

A pharmaceutical composition to be administered systemically may be confected as a powder, pill, tablet or the like, or as a solution, emulsion, suspension, aerosol, syrup or elixir suitable for oral or parenteral administration or inhalation.

For solid dosage forms or medicaments, non-toxic solid carriers include, but are not limited to, pharmaceutical grades of mannitol, lactose, starch, magnesium stearate, sodium saccharin, the polyalkylene glycols, talcum, cellulose, glucose, sucrose and magnesium carbonate. The solid dosage forms may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the technique described in U.S. Pat. No. 4,256,108, U.S. Pat. No. 4,166,452, and U.S. Pat. No. 4,265,874 to form osmotic therapeutic tablets for control release. Liquid pharmaceutically administrable dosage forms can, for example, comprise a solution or suspension of one or more of the presently useful compounds and optional pharmaceutical adjutants in a carrier, such as for example, water, saline, aqueous dextrose, glycerol, ethanol and the like, to thereby form a solution or suspension. If desired, the pharmaceutical composition to be administered may also contain minor amounts of nontoxic auxiliary substances such as wetting or emulsifying agents, pH buffering agents and the like. Typical examples of such auxiliary agents are sodium acetate, sorbitan monolaurate, triethanolamine, sodium acetate, triethanolamine oleate, etc. Actual methods of preparing such dosage forms are known, or will be apparent, to those skilled in this art; for example, see Remington's Pharmaceutical Sciences, Mack Publishing Company, Easton, Pa., 16th Edition, 1980. The composition of the formulation to be administered, in any event, contains a quantity of one or more of the presently useful compounds in an amount effective to provide the desired therapeutic effect.

Parenteral administration is generally characterized by injection, either subcutaneously, intramuscularly or intravenously. Injectables can be prepared in conventional forms, either as liquid solutions or suspensions, solid forms suitable for solution or suspension in liquid prior to injection, or as emulsions. Suitable excipients are, for example, water, saline, dextrose, glycerol, ethanol and the like. In addition, if desired, the injectable pharmaceutical compositions to be administered may also contain minor amounts of non-toxic auxiliary substances such as wetting or emulsifying agents, pH buffering agents and the like.

### Methods of Treatment

The pharmaceutical compositions of the invention may be used to treat motility disorders. To “treat,” as used here, means to deal with medically. It includes administering agents of the invention to prevent the onset of a condition, ameliorate its symptoms, address its cause, or to prevent its reoccurrence. All these things fall within the meaning of “treating.”

One can treat, according to the method of the invention, motility disorders or their symptoms by administering to a patient a combination of one or more of an acid reducer and one or more of an alpha-2B receptor agonist. The foregoing agents may be administered together, but one can also administer these compounds separately, administering one immediately after the other, or administering one within a short interval after the other (e.g., 5-15 minutes, or 15-30 minutes, or 30 minutes-1 hour), or administering one within a longer interval after the other (e.g., 1-2 hours, 2-4 hours, 4-6 hours, 6-12 hours, or 12-24 hours). One can also administer one compound more frequently than another, administering, for example, an acid reducer one or more times daily and an alpha-2B receptor agonist two or more times daily (or vice versa).

The acid reducers and alpha-2B receptor agonists of the invention may be administered in a single formulation (e.g., a single pill or injection), or may be administered separately, each in its own formulation (e.g., a proton pump inhibitor orally once daily and an alpha-2B receptor agonist twice daily via injection).

A patient may be administered the usual course of acid reducer and the usual course of alpha-2 agonist, but a patient may also receive a reduced course of one or the other therapy or of both therapies (that is, a patient may take a lower dose than is usually prescribed or may take it for a shorter duration).

An “effective dose,” means a dose which reduces discomfort in a patient to tolerable levels.

### Dose

Pharmaceutical compositions of the invention may be formulated such that a patient receives a dose of an acid reducer that is usually effective, when administered separately, to treat a motility disorder, and a dose of an alpha-2B receptor agonist that is usually effective, when administered separately, to treat a motility disorder. But the pharmaceutical compositions of the invention may also be formulated such that doses of each compound may be those that are ineffective or minimally effective when the compounds are administered alone. This allows one to administer to a patient a formulation of the invention that is as effective as a larger dose of an acid reducer or alpha-2B receptor agonist when administered alone, but less likely to lead to side effects. This does not mean, however, that formulations of the invention comprise acid reducers and alpha-2B receptor agonists in only such doses which are, when administered alone, minimally effective: a patient with severe discomfort may require a high dose of either component of the formulation, but is still likely to experience enhanced symptom relief (as compared to the relief the patient would experience were he administered a high dose of either component of the invention alone).

The precise dose and frequency of administration depends on the severity and nature of the patient's condition, on the manner of administration, on the potency and pharmacodynamics of the particular compound employed, and on the judgment of the prescribing physician. Determining dose is a routine matter that is well within the capability of someone of ordinary skill in the art. The usual effective dose of acid reducers are set forth in the tables below as a guide.

Although most of the antacids listed in Table 2 are formulated in tablets, any formulation known in the art may be used.

TABLE 2
usual effective doses of some common antacids

| ACID REDUCER | ADULT DOSE |
|---|---|
| Aluminum Hydroxide (300 mg) + | 1-2 tablets as needed |
| Magnesium Hydroxide (150 mg) | |
| combination tablet | |
| Aluminum Hydroxide (200 mg) + | 1-2 tablets as needed |
| Magnesium Hydroxide (200 mg) | |
| combination tablet | |
| Calcium carbonate | 400 mg-800 mg as needed |
| Calcium carbonate (700 mg) + magnesium | 2-4 tablets between meals and |
| hydroxide (300 mg) | at bedtime |
| combination tablet | |
| Citric acid (1,000 mg) + potassium | 1-2 tablets every four hours |
| bicarbonate (344 mg) + sodium | |
| bicarbonate (1,050 mg) combination | |
| tablet | |
| Bismuth subsalicylate | 262 mg-524 mg every 30 |
| | minutes to 1 hour, as needed |

TABLE 3
usual effective doses of some common histamine H₂ blockers

| ACID REDUCER | ADULT DOSE |
|---|---|
| Ranitidine | 75 or 150 mg twice daily |
| Cimetidine | 400 mg or 800 mg once daily |
| Famotidine | 10 or 20 mg once or twice |
| | daily or 40 mg once daily |
| Nizatidine | 150 mg twice daily or 300 mg |
| | once daily |

TABLE 4
usual effective doses of some common proton pump inhibitors

| ACID REDUCER | ADULT DOSE |
|---|---|
| Omeprazole | 20 mg once daily |
| Lansoprazole | 15 or 30 mg once or twice |
| | daily |
| Esomeprazole | 20 or 40 mg once daily |
| Pantoprazole | 40 mg once daily |
| Rabeprazole | 20 mg once daily |

What is claimed is:

1. A pharmaceutical composition comprising an acid reducer and an alpha-2B receptor agonist.

2. The composition of claim 1, wherein the acid reducer is selected from the group consisting of an antacid, a proton pump inhibitor, and a histamine H₂ antagonist.

3. The composition of claim 2, wherein the histamine H₂ antagonist is selected from the group consisting of cimetidine, famotidine, nizatidine, and ranitidine.

4. The composition of claim 2, wherein the proton pump inhibitor is selected from the group consisting of esomeprazole, lansoprazole, omeprazole, pantoprazole, and rabeprazole.

5. A method of treating a gastrointestinal motility disorder, the method comprising the step of administering to a patient in need of such treatment one or more of an acid reducer and one or more of an alpha-2B receptor agonist.

6. The method of claim 5, wherein the gastrointestinal motility disorder is selected from the group consisting of achalasia, Barrett's syndrome, biliary dyskinesia, Crohn's disease, chronic intestinal pseudo-obstruction, colonic inertia, constipation, cyclic vomiting syndrome, diarrhea, diffuse esophageal spasm, dumping syndrome, dyspepsia, dysphagia, encopresis, fecal incontinence, functional abdominal pain (e.g., chronic proctalgia, epigastric pain syndrome, functional abdominal pain syndrome, proctalgia fugax), functional biliary disorders (e.g., functional biliary SO disorder, functional gallbladder disorder, functional pancreatic SO disorder, functional sphincter of Oddi disorder), functional bowel outlet obstruction, functional dyspepsia disorders (e.g., epigastric pain syndrome, functional dyspepsia, postprandial distress syndrome), functional esophogeal disorders (e.g., functional chest pain of presumed esophogeal origin, functional dysphagia, functional heartburn, globus), functional fecal retention, gastroesophageal reflux disease (GERD), gastroparesis, gastritis, gastropathy, Hirschprung's disease, hypercontractile motility, hypermotility, hypertensive lower esophageal sphincter, hypomotility, intestinal obstruction, irritable bowel syndrome, ischemia, megacolon, non-erosive reflux disease, pancreatitis, pelvic floor dysfunction, short bowel syndrome, small bowel bacterial overgrowth, small bowel intestinal motility disorder, superior mesenteric artery syndrome, ulcerative colitis, and volvulus.

7. The method of claim 5, wherein the gastrointestinal motility disorder is selected from the group consisting of altered bowel habit, belching, bloating, blood or mucus in the stool, diarrhea, dyspepsia, dysphagia, flatulence, globus, hoarseness of voice, loss of appetite, nausea, pain in the chest, pain in the colon, pain in the abdomen, pyrosis, regurgitation, sore throat, trapped gas, and uncomfortable fullness after meals.

8. The method of claim 5, wherein the acid reducer is selected from the group consisting of an antacid, a histamine H₂ antagonist, and a proton pump inhibitor.

9. The method of claim 8, wherein the histamine H₂ antagonist is selected from the group consisting of cimetidine, famotidine, nizatidine, and ranitidine.

10. The method of claim 8, wherein the proton pump inhibitor is selected from the group consisting of esomeprazole, lansoprazole, omeprazole, pantoprazole, and rabeprazole.

11. The method of any one of claims 5-10, wherein the acid reducer and the alpha-2B receptor agonist are administered in a single formulation.

12. The method of any one of claims 5-10, wherein a first formulation comprising the acid reducer and a second formulation comprising the alpha-2B receptor agonist are administered at the same time.

13. The method of any one of claims 5-10, wherein a first formulation comprising the acid reducer and a second formulation comprising the alpha-2B receptor agonist are administered at different times.

14. The method of any one of claims 5-10, wherein a first formulation comprising the acid reducer is administered once daily and a second formulation comprising the alpha-2B receptor agonist is administered once daily.
